(19) United States
(12) Patent Application Publication
(10) Pub. No.: US 2025/0305674 A1
(43) Pub. Date: Oct. 2, 2025

(54) PAIR OF SUPPORT PLATES FOR TUBES IN A REACTOR VESSEL

(71) Applicant: L'Air Liquide, Société Anonyme pour l'Etude et l'Exploitation des Procédés Georges Claude, Paris (FR)

(72) Inventors: Tobias Oelmann, Bad Vilbel (DE); Michael Strozyk, Offenbach (DE); David Personne, Frankfurt am Main (DE); Abdullah Enes Tercan, Frankfurt am Main (DE); Veronika Gronemann, Frankfurt am Main (DE)

(21) Appl. No.: 19/090,912

(22) Filed: Mar. 26, 2025

(30) Foreign Application Priority Data
Mar. 28, 2024 (EP) 24167624

(51) Int. Cl. F22B 37/20; F22B 1/02

(52) U.S. Cl. CPC F22B 37/205; F22B 1/023

(57) ABSTRACT

A device (1.1; 1.2) with a reactor vessel (2), a tube bundle (3) of multiple tubes (4), a first support plate (5) and a second support plate (6), wherein the tube bundle (3) is located in the reactor vessel (2), wherein the tube bundle (3) comprises multiple first tube groups (7) and multiple second tube groups (8), wherein the first support plate (5) and the second support plate (6) are disposed in the reactor vessel (2) transversely to a longitudinal axis (9) of the reactor vessel (2), wherein the first support plate (5) is offset from the second support plate (6) along the longitudinal axis (9) of the reactor vessel (2).

### CROSS REFERENCE TO RELATED APPLICATIONS

This application claims the benefit of priority under 35 U.S.C. § 119 (a) and (b) to European Patent Application No. EP24167624, filed Mar. 28, 2024, the entire contents of which are incorporated herein by reference.

### BACKGROUND

The invention relates to an assembly for methanol synthesis and to a device that can be part of such an assembly. The invention also relates to the use of the device and to a process for methanol synthesis using the device.

Processes for the industrial preparation of methanol by heterogeneously catalysed conversion of synthesis gas in suitable synthesis reactors are known. Synthesis gases may be different gas mixtures which include hydrogen and carbon dioxide. The reactor is usually in the form of an upright fixed-tube heat exchanger.

Two-stage processes for preparing methanol are also known. Synthesis gas is fed to a water-cooled reactor and then to a gas-cooled reactor. A copper-based solid-bed catalyst is used to convert the synthesis gas to methanol.

In the case of a water-cooled reactor, the catalyst is inside the tubes, surrounded by water or steam on the shell side. The tubes are mechanically fixed via tube plates and supporting metal sheets in the reactor.

The cooling in the water-cooled reactor is performed via heat being released into the water, whereupon steam can be produced. The steam-water mixture rises on the tubes. The supporting metal sheets must ensure the fixing of the tubes. Supporting metal sheets inserted in offset fashion in the reactor vessel, such that the coolant flows in a meandering course in the reactor vessel around the supporting metal sheets, are known.

Depending on the selected geometries of the heat-transferring components and of the mechanical elements of the coolant side, a pressure drop is generated on the coolant side in the steam. It is primarily long reactor tubes which result in a higher pressure drop.

Frequently, the external dimensions of the reactor are restricted for transport, and this leads to long, slender reactors. However, the consequence of this is an elevated pressure drop on the coolant side. The long shape of the reactor means further support plates are required to avoid the tubes sagging when they are being transported, and also avoid them bowing, bending and swaying when in operation.

### SUMMARY

An object of the present invention, proceeding from the described prior art, is to reduce the hindering effect of the support plates and at the same time prevent the tubes from bending, bowing or swaying.

This object is achieved by the subject matter of the independent claims. Further advantageous configurations are specified in the dependent claims. The features set out in the claims and in the description may be combined with one another in any technologically appropriate way.

The invention sets out a device which comprises a reactor vessel, a tube bundle of multiple tubes, a first support plate and a second support plate. The tube bundle is located in the reactor vessel. The tube bundle comprises multiple first tube groups and multiple second tube groups. The first support plate and the second support plate are disposed in the reactor vessel transversely to a longitudinal axis of the reactor vessel. The first support plate is offset from the second support plate along the longitudinal axis of the reactor vessel.

Each of the tubes of the first tube groups is routed through a respective tube opening of the first support plate, and the first support plate has multiple fluid-exchange apertures. Each of the second tube groups is routed through a respective one of the apertures in the first support plate.

Each of the tubes of the second tube groups is routed through a respective tube opening of the second support plate, and the second support plate has multiple fluid-exchange apertures. Each of the first tube groups is routed through a respective one of the apertures in the second support plate.

The first support plate supports the tubes of the first tube groups in the tube openings of the first support plate transversely to the longitudinal direction of the tubes and the second support plate supports the tubes of the second tube groups in the tube openings of the second support plate transversely to the longitudinal direction of the tubes.

The device is preferably in the form of a reactor. The device may be designed to carry out a chemical reaction. The chemical reaction may be an exothermic or endothermic reaction. The device is particularly suitable for methanol synthesis. However, the advantages described here can also be achieved for numerous other chemical reactions. The advantages can even be achieved if the device is not used as a reactor and no chemical reaction proceeds in the device. The device may in general be in the form of a heat exchanger.

The device comprises a reactor vessel and a tube bundle of multiple tubes in the reactor vessel. A first medium can flow through the tubes. Outside the tubes there may be a second medium in the reactor vessel. The second medium can flow through intermediate spaces between the tubes. Between the first medium and the second medium, an exchange of heat can take place. The first medium and the second medium can flow in opposite directions. In this case, the device is operated in a counter-current configuration. The first medium and the second medium can, however, also flow in the same direction. In the case of methanol synthesis, the first medium may contain for example the reaction reactants and the second medium may be a cooling medium, or vice versa.

The reactor vessel is preferably an elongate hollow body. The reactor vessel may be a cylindrical metal vessel suitable for encompassing the tube bundle. The reactor vessel may have ports and connections for fluidically connecting the tube bundle. Furthermore, the reactor vessel may have ports and connections so that a cooling medium or other medium can be introduced into and discharged from the reaction vessel into or from a shell space outside and between the tubes. The shell space may be fluidically connected.

The tube bundle comprises multiple tubes. The tube bundle preferably comprises at least 18 tubes. The tube bundle preferably comprises at least 100 tubes. The tube bundle particularly preferably comprises even at least 1000 tubes.

The tube bundle comprises multiple first tube groups and multiple second tube groups.

In particular, the first tube groups may each comprise between 9 and 36 tubes and/or the second tube groups may each comprise between 9 and 36 tubes.

The tubes may be elongate cylindrical hollow bodies with a uniform wall thickness. The wall thickness of the tubes is preferably at most 5 mm, more preferably at most 3 mm and particularly preferably at most 0.1 mm. In particular, the wall thickness of the tubes can correspond to a wall thickness which is standard for the application. The tubes may be made in particular of a metallic material. The material preferably conducts heat.

Some of the tubes or all of the tubes preferably contain a catalyst. In the case of methanol synthesis, it is thus possible for example to conduct the reaction reactants through the tubes and react them with the catalyst in the tubes so as to form methanol.

The reaction reactants may be provided in particular in the form of a synthesis gas. Preferred reaction reactants are hydrogen, carbon monoxide and carbon dioxide. A mixture of hydrogen and carbon monoxide is preferred. A mixture of hydrogen and carbon dioxide is particularly preferred. Furthermore, the synthesis gas may contain inert gases. In particular, the synthesis gas may contain methane as inert gas. The synthesis gas preferably contains nitrogen as inert gas. The synthesis gas can be conducted into the tubes on an inlet side and react with the catalyst in the tube. In the case of an exothermic reaction, heat generated during the reaction can be released via the tube shell. A coolant can discharge the heat, preferably by convection, from the shell side. The reaction product formed in the tube can, together with the remaining synthesis gas, be discharged on the outlet side.

As an alternative, a synthesis gas with the reaction reactants can also be conducted through the reaction vessel outside the tubes. In particular in this case, a catalyst bed may be provided on the shell side in the shell space. The synthesis gas can then be passed through the catalyst bed. A coolant can be conducted into the tubes on an inlet side. The heat generated by the reaction can be transferred to the coolant in the tubes via the shell side of the tubes. The heated coolant can be discharged on an outlet side of the tubes.

Furthermore, the device comprises a first support plate and a second support plate. The first support plate and/or the second support plate may be in the form of a metal sheet. The first support plate and/or the second support plate preferably have a thickness of at least 7 mm. The thickness of the first support plate and/or of the second support plate is preferably at most half of a diameter of the tubes. The optimum thickness of the support plate can also be ascertained via calculations. If not all the tubes have the same diameter, the thickness of the first support plate and/or of the second support plate is preferably at most half of the largest diameter of the tubes of the tube bundle.

The first support plate and the second support plate are disposed in the reactor vessel transversely to a longitudinal axis of the reactor vessel. In particular, the first support plate and the second support plate may be fastened in place in the reactor vessel.

The longitudinal axis of the reactor vessel is preferably aligned along the elongate direction of the hollow body. In particular, the first support plate and/or the second support plate is disposed in the reactor vessel perpendicularly in relation to the longitudinal axis of the reactor vessel. The reactor vessel and/or the tube bundle is preferably vertically aligned. The “and” versions are preferred.

The first support plate is offset from the second support plate along the longitudinal axis of the reactor vessel.

The first support plate is preferably at a spacing of at least 100 mm from the second support plate along the longitudinal axis of the reactor vessel. The first support plate is more preferably at a spacing of at least 350 mm from the second support plate along the longitudinal axis of the reactor vessel. The first support plate is particularly preferably at a spacing of at least 700 mm from the second support plate along the longitudinal axis of the reactor vessel.

The first support plate is preferably at a spacing of at most 2000 mm from the second support plate along the longitudinal axis of the reactor vessel.

Each of the tubes of the first tube groups is routed through a respective tube opening of the first support plate, and the first support plate has multiple fluid-exchange apertures. Each of the second tube groups is routed through a respective one of the apertures in the first support plate. Each of the tubes of the second tube groups is routed through a respective tube opening of the second support plate, and the second support plate has multiple fluid-exchange apertures. Each of the first tube groups is routed through a respective one of the apertures in the second support plate.

In particular, the tube openings of the first support plate are assigned to the tubes of the first tube groups and the tube openings of the second support plate are assigned to the tubes of the second tube groups.

The first support plate and the second support plate may adversely affect the fluid exchange in the shell space of the reactor vessel. In order to keep this effect as small as possible, the first support plate and the second support plate each have multiple apertures. The fluid exchange takes place in particular between reactor-vessel regions between which the first support plate and the second support plate are located.

Each of the tubes of the tube bundle belongs either to exactly one of the first tube groups, to exactly one of the second tube groups, or to none of the tube groups. Even if there are tubes that belong to none of the tube groups, there is in any case no tube which belongs to more than one of the tube groups at the same time. The tube groups are defined only by the properties described herein. It is not necessary for the assignment of the tubes to the tube groups to be evident in addition on structural features of the device.

The tubes of all the first tube groups are routed through a respective tube opening of the first support plate. Each tube has a dedicated tube opening. As a result, the tubes of the first tube group are supported by the first support plate. The tubes of the first tube groups are moreover routed through one of the apertures of the second support plate. Each of the first tube groups is in the process routed through a respective one of the apertures in the second support plate. Each first tube group has a dedicated aperture. All the tubes of a first tube group are routed through this aperture together. The first tube groups thus each comprise all the tubes that are routed through one of the cutouts of the second support plate together. The tubes of the first tube groups are in general not supported by the second support plate. Instead, these tubes pass through the second support plate merely by being routed through the corresponding aperture. The apertures in the second support plate are used for fluid exchange.

The tubes of all the second tube groups are routed through a respective tube opening of the second support plate. Each tube has a dedicated tube opening. As a result, the tubes of the second tube group are supported by the second support plate. The tubes of the second tube groups are moreover routed through one of the apertures of the first support plate. Each of the second tube groups is in the process routed through a respective one of the apertures in the first support plate. Each second tube group has a dedicated aperture. All the tubes of a second tube group are routed through this aperture together. The second tube groups thus each comprise all the tubes that are routed through one of the cutouts of the first support plate together. The tubes of the second tube groups are in general not supported by the first support plate. Instead, these tubes pass through the first support plate merely by being routed through the corresponding aperture. The apertures in the first support plate are used for fluid exchange. If there is a tube which belongs neither to the first tube group nor to the second tube group, this tube can be routed for example both in the first support plate and in the second support plate through a respective tube opening. Such a tube is then supported both by the first support plate and by the second support plate.

Each of the tubes is thus supported either at least by the first support plate or at least by the second support plate. The interaction of the first support plate and the second support plate thus brings about the desired support of all the tubes. The first support plate and the second support plate may be construed as a support unit in this respect. The advantages described herein are already achieved if the device has such a support unit. However, it is also possible and even preferable for the device to have multiple support units, which each have a first support plate and a second support plate. The support units are preferably each designed as described herein.

The apertures in the first support plate and in the second support plate preferably do not lie congruently one above another. The apertures may, however, nonetheless partially overlap. In an intermediate space between the first support plate and the second support plate, the fluid can flow from the apertures of the first support plate to the apertures of the second support plate, or vice versa. This can create meandering flows of the fluid that travel partially transversely to the longitudinal axis.

A free flow cross section in the reactor vessel can be determined by the apertures in the first support plate and in the second support plate. The free flow cross section is that cross section of a tube or channel through which a medium flows. The free flow cross section thus denotes an area through which a medium can flow. The greater the smallest overall area of all the apertures of the first support plate or of the second support plate is, the greater the free flow cross section is.

The first support plate supports the tubes of the first tube groups in the tube openings of the first support plate transversely to the longitudinal direction of the tubes and the second support plate supports the tubes of the second tube groups in the tube openings of the second support plate transversely to the longitudinal direction of the tubes. This has the advantage that the tubes of the tube bundle can individually thermally expand, but vibrations caused by certain flow states and bending of the tubes can be avoided. A further advantage is that all the tubes of the tube bundle are supported by a first support plate together with a second support plate.

The tubes of the tube bundle are preferably not axially fixed in place in the respective tube opening of the first support plate and of the second support plate. The tubes of the first tube group are in this case axially movable in the axial direction through a respective tube opening of the first support plate. The tubes of the second tube group are in this same case axially movable in the axial direction through a respective tube opening of the second support plate. Locally different temperatures can result in locally different expansions of the tubes. Since the tubes are axially movable, individual tubes are not disrupted in their individual expansion by one of the support plates. Movements transverse to the longitudinal axis are, however, minimized.

The shape of the first support plate and of the second support plate can be adapted to that of the reactor vessel. The first support plate and/or the second support plate preferably has a rectangular shape with rounded corners. The first support plate and/or the second support plate particularly preferably has an elliptical shape, in particular a circular shape. The “and” versions are preferred.

The device has the advantage that the hindering effect of the first support plate and of the second support plate is minimized and at the same time the tubes are prevented from bending, bowing or swaying during transport or during operation.

In a preferred embodiment, the apertures in the first support plate and/or the apertures in the second support plate are diamond-shaped.

The apertures of the first support plate may, given 9 tubes per second tube group, comprise 3 by 3 tubes in a diamond shape. The apertures of the second support plate may, given 9 tubes per first tube group, comprise 3 by 3 tubes in a diamond shape.

The apertures of the first support plate may, given 16 tubes per second tube group, comprise 4 by 4 tubes in a diamond shape. The apertures of the second support plate may, given 16 tubes per first tube group, comprise 4 by 4 tubes in a diamond shape.

The apertures of the first support plate may, given 25 tubes per second tube group, comprise 5 by 5 tubes in a diamond shape. The apertures of the second support plate may, given 25 tubes per first tube group, comprise 5 by 5 tubes in a diamond shape.

The apertures of the first support plate may, given 36 tubes per second tube group, comprise 6 by 6 tubes in a diamond shape. The apertures of the second support plate may, given 36 tubes per first tube group, comprise 6 by 6 tubes in a diamond shape.

The advantage of this embodiment is that the free flow cross section is larger owing to the diamond-shaped cutouts and the flow through the first flow plate and through the second flow plate is disrupted as little as possible.

In a further, preferred embodiment, a respective plurality of first tube groups are next to one another in a first row, wherein a respective plurality of second tube groups are next to one another in an adjacent second row, wherein first and second rows alternate.

A perspective in which only the apertures of the first and the second support plate are considered, and the first support plate and second support plate are superimposed in a plane, is referred to as superimposed perspective in the following text.

From a superimposed perspective, accordingly, a respective plurality of apertures of the second support plate are next to one another in a first row, wherein a respective plurality of apertures of the first support plate are next to one another in an adjacent second row, wherein first and second rows alternate. This may also be referred to as a pattern.

This embodiment has the advantage of reducing the pressure drop owing to flow resistances on the first support plate and on the second support plate. This is because the fluid is deflected to a lesser extent between the first support plate and the second support plate in this embodiment.

In a further, preferred embodiment, the apertures in the first support plate each have a hexagonal shape and/or the apertures in the second support plate each have a hexagonal shape.

In particular, the respective hexagonal shape may be elongate, with 2 short, opposite sides and 4 long sides. This shape can also be described as a truncated diamond. The first tube groups and second tube groups preferably alternate in a chequerboard pattern.

In particular, a second tube group may be surrounded by four first tube groups and/or a first tube group may be surrounded by four second tube groups.

Accordingly, from a superimposed perspective, a respective plurality of apertures of the second support plate together with a plurality of apertures of the first support plate may alternate in a chequerboard pattern.

The advantage of this embodiment is that the first and the second support plate cause a lower pressure drop, because in this embodiment a shorter flow path is established for the flow. Furthermore, a symmetrical flow forms between the support plates, and this in turn manifests itself in a uniform force being exerted on the support plates.

In a further embodiment, a respective first tube group may be surrounded by six second tube groups. Accordingly, from a superimposed perspective, six apertures of the first support plate for passage of the six second tube groups are disposed around the one aperture of the second support plate for passage of the one first tube group. It is also conceivable for a respective second tube group to be surrounded by six first tube groups. Accordingly, from a superimposed perspective, six apertures of the second support plate for passage of the six first tube groups are disposed around the one aperture of the first support plate for passage of the one second tube group.

The advantage of this embodiment is that the first and the second support plate cause a lower pressure drop owing to the fluid exchange, because in this embodiment a shorter flow path is established for the flow. Furthermore, a symmetrical flow forms between the support plates, and this in turn manifests itself in a uniform force being exerted on the support plates.

This embodiment also has the advantage that the loading exerted by the flow on the edges of the apertures can be reduced, as a result of which the pressure drop and the mechanical loading exerted on the support plates can be further reduced.

In a further, preferred embodiment, the first support plate has, in addition to the apertures, fluid-exchange cutouts and/or the second support plate has, in addition to the apertures, fluid-exchange cutouts.

This embodiment has the advantage that the pressure drop is reduced by flow resistances.

In a further preferred embodiment, the first support plate, for at least some of the cutouts of the first support plate, forms a respective divider in each case between the cutout and the closest tube opening of the first support plate to the cutout, and wherein the dividers have a respective minimum width which is the same for at least some of the dividers, and/or the second support plate, for at least some of the cutouts of the second support plate, forms a respective divider in each case between the cutout and the closest tube opening of the second support plate to the cutout, and wherein the dividers have a respective minimum width which is the same for at least some of the dividers.

The first support plate and the second support plate should on the one hand be stable enough to sufficiently support the tubes. This can be achieved by the dividers having a sufficient width. On the other hand, the support plate should adversely affect the fluid exchange as little as possible. This can be achieved by the dividers being kept as small as possible. Assessing the two aforementioned conditions will make it possible to determine how wide a divider should optimally be at its narrowest point, and thus how large the minimum width of a divider should optimally be. In the present embodiment, at least some of the dividers, preferably even all the dividers, have the same minimum width. In this way, the best possible compromise between stability and fluid exchange can be achieved for all these dividers.

In a further, preferred embodiment, at least some of the cutouts of the first support plate each lie centrally between three tube openings of the first support plate that are adjacent to one another in pairs, and/or at least some of the cutouts of the second support plate each lie centrally between three tube openings for the second tube groups of the second support plate that are adjacent to one another in pairs.

The cutouts of the support plates may for example each lie centrally between three adjacent tube openings, the centres of these three tube openings preferably forming an equilateral triangle.

In a further, preferred embodiment, at least some of the cutouts of the first support plate are passage bores and/or at least some of the cutouts of the second support plate are passage bores.

The advantage of passage bores is that the first and the second support plate can be produced particularly easily and have a very high strength and stiffness. In a further, preferred embodiment, at least some of the cutouts of the first support plate are star-shaped and/or at least some of the cutouts of the second support plate are star-shaped.

A star-shaped cutout is to be understood in the mathematical sense in this case. A respective cutout is star-shaped when there is a point, what is referred to as the centre of the star, from which all other points of the cutout are visible. That means that every line connecting a point to the centre of the star lies completely in the cutout. The centre of the star can, figuratively speaking, “see” all sides of the cutout.

At least some of the cutouts of the first support plate and at least some of the cutouts of the second support plate preferably have the shape of triangular holes. A triangular hole in this case means a three-sided hole with rounded corners and concave sides. A triangular hole is star-shaped in the sense of the term star-shaped that is used herein.

The advantage of this embodiment is that the area of the cutout can be increased without further modifications. Figuratively speaking, the available space between the tube openings can be utilized better. This makes it possible to further reduce the hindering effect of the first support plate and of the second support plate.

In a further, preferred embodiment, the first support plate has intermediate regions, which are each formed between three tube openings that are adjacent to one another in pairs, and wherein at least some of the cutouts of the first support plate are restricted to a respective one of the intermediate regions. In addition or alternatively, the second support plate has intermediate regions, which are each formed between three tube openings that are adjacent to one another in pairs, and wherein at least some of the cutouts of the second support plate are restricted to a respective one of the intermediate regions.

The advantage of this embodiment is that the area of the cutout can be increased given otherwise unchanged conditions. Figuratively speaking, the intermediate region between the tube openings can be utilized better. This makes it possible to further reduce the hindering effect of the support plates.

In a further, preferred embodiment, the first support plate has intermediate regions, which are each formed between three tube openings that are adjacent to one another in pairs, and wherein at least some of the cutouts of the first support plate each extend across two of the intermediate regions. In addition or as an alternative, the second support plate has intermediate regions, which are each formed between three tube openings that are adjacent to one another in pairs, and wherein at least some of the cutouts of the second support plate each extend across two of the intermediate regions.

This configuration can be obtained by removing the respective connecting divider between two adjacent intermediate regions. In this configuration, for example three cutouts may be formed around a tube opening. For each cutout, two respective adjacent intermediate regions may be connected. Each cutout can have four adjacent tube openings. This can be applied to the first and/or the second support plate.

The advantage of this embodiment is that the hindering effect of the support plate can be reduced. Furthermore, the support plates can be made more lightweight by this embodiment.

A further aspect of the invention sets out an assembly for methanol synthesis. The assembly comprises the described device.

The described advantages and features of the device can be applied and transferred to the assembly.

To produce methanol, a synthesis gas can be converted by reaction over catalysts to yield methanol. The synthesist gas can contain reaction reactants for the methanol synthesis.

Besides the described device, the assembly preferably comprises a source for reaction reactants for the methanol synthesis. The assembly may also comprise multiple devices designed as described and arranged for example in series.

A further aspect of the invention sets out a process. In the process, reaction reactants for the methanol synthesis are conducted through the tubes of the tube bundle. A cooling medium is conducted through the reactor vessel outside the tubes of the tube bundle. The cooling medium is preferably liquid water and/or steam.

In an alternative embodiment of the process, the reaction reactants for the methanol synthesis are conducted through the reactor vessel around the tubes of the tube bundle. A cooling medium is then conducted through the tubes of the tube bundle. The cooling medium is preferably gaseous.

The described advantages and features of the device can be applied and transferred to the process, and vice versa. The device is preferably designed for operation according to the described process. The process is preferably performed with the device.

A further aspect of the invention sets out the use. The described device is used for methanol synthesis. Reaction reactants are converted to methanol in the device.

The described advantages and features of the device, the assembly and the method can be applied and transferred to the use, and vice versa.

The device is preferably used for water-cooled methanol synthesis. As an alternative, it is preferred to use the device for gas-cooled methanol synthesis.

In water-cooled methanol synthesis, the tubes are cooled with water, while a synthesis gas is converted to methanol in the tubes. The water discharges the heat from the tubes and partially evaporates. The rising bubbles of steam can flow through the apertures of the first support plate and through the apertures of the second support plate, or vice versa. In particular, the rising bubbles of steam can flow through the cutouts of the first support plate and through the cutouts of the second support plate, or vice versa.

In gas-cooled methanol synthesis, the tubes are cooled by a cooler gas in the tubes. The synthesis gas is converted to methanol outside the tubes. The heat is transferred to the tubes and discharged by the gas in the tubes. The synthesis gas can flow through the apertures of the first support plate and through the apertures of the second support plate, or vice versa. In particular, the synthesis gas can flow through the cutouts of the first support plate and through the cutouts of the second support plate, or vice versa.

### BRIEF DESCRIPTION OF THE DRAWINGS

The invention is elucidated in detail hereinafter with reference to the figures. The figures show a particularly preferred exemplary embodiment, but the invention is not limited thereto. The figures and the size ratios represented therein are merely schematic. In the figures:

FIG. 1: shows a schematic view of an assembly according to the invention for methanol synthesis,

FIG. 2: shows a schematic plan view of a first configuration of a first support plate, as can be used in the assembly from FIG. 1,

FIG. 3: shows a schematic plan view of a first configuration of a second support plate, as can be used in the assembly from FIG. 1,

FIG. 4: shows a cutout of a first support plate in a first configuration from FIG. 2,

FIG. 5: shows a cutout of a second support plate in a first configuration from FIG. 3,

FIG. 6: shows a schematic plan view of a first support plate with apertures and of a second support plate with apertures from a superimposed perspective, in a first configuration from FIG. 2 to FIG. 5, as can be used in the assembly from FIG. 1,

FIG. 7: shows a view of a detail of the first configuration of the apertures of the first and second support plates from a superimposed perspective from FIG. 6,

FIG. 8: shows a view of a detail of a second configuration of the apertures of the first support plate and of the second support plate from a superimposed perspective, as can be used in the assembly from FIG. 1,

FIG. 9: shows a view of a detail of a third configuration of the apertures of the first support plate and of the second support plate from a superimposed perspective, as can be used in the assembly from FIG. 1,

FIG. 10: shows a view of a detail of a fourth configuration of the first and the second support plate, as can be used in the assembly from FIG. 1,

FIG. 11: shows a view of a detail of a fifth configuration of the first and the second support plate, as can be used in the assembly from FIG. 1,

FIG. 12: shows a view of a detail of a sixth configuration of the first and the second support plate, as can be used in the assembly from FIG. 1,

FIG. 13: shows a view of a detail of a seventh configuration of the apertures and the cutouts of the first support plate, as can be used in the assembly from FIG. 1,

FIG. 14: shows a view of a detail of a seventh configuration of the apertures and the cutouts of the first support plate from FIG. 13.

### DESCRIPTION OF THE PREFERRED EMBODIMENTS

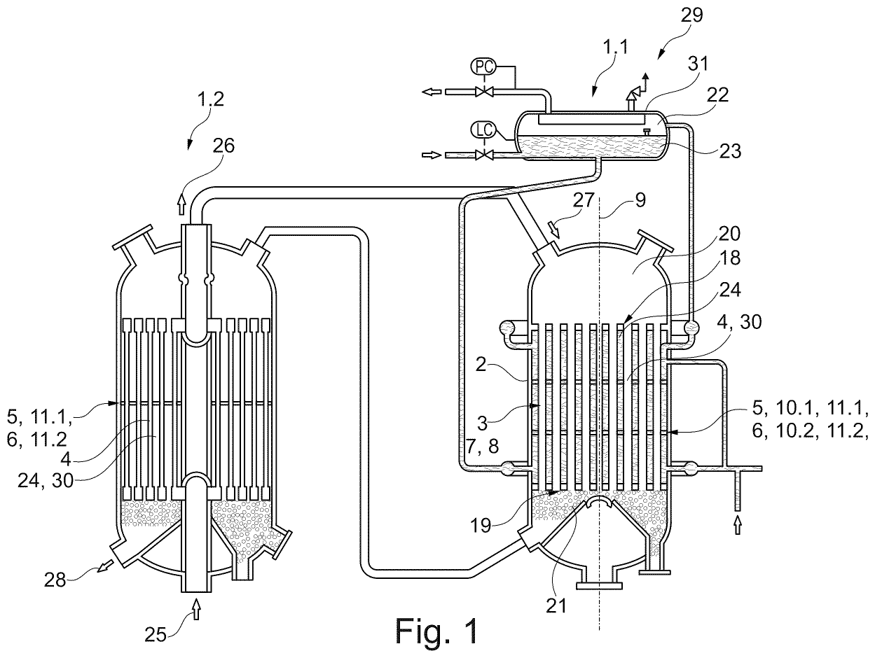

FIG. 1 shows a schematic view of an assembly 29 for methanol synthesis. Synthesis gas is conducted through two devices 1.1; 1.2 connected in series. The devices 1.1; 1.2 are used for methanol synthesis.

Pre-heated synthesis gas is fed through the gas inlet 27 of the first device 1.1.

The first device 1.1 comprises a reactor vessel 2, a tube bundle 3 of multiple tubes 4, a first support plate 5 and a second support plate 6. The tube bundle 3 is located in the reactor vessel 2. The tube bundle 3 comprises multiple first tube groups 7 and multiple second tube groups 8. The first support plate 5 and the second support plate 6 are disposed in the reactor vessel 2 transversely to a longitudinal axis 9 of the reactor vessel 2. The first support plate 5 is offset from the second support plate 6 along the longitudinal axis 9 of the reactor vessel 2.

Each of the tubes 4 of the first tube groups 7 is routed through a respective tube opening 10.1 of the first support plate 5, and the first support plate 5 has multiple fluid-exchange apertures 11.1. Each of the second tube groups 8 is routed through a respective one of the apertures 11.1 in the first support plate 5.

Each of the tubes 4 of the second tube groups 8 is routed through a respective tube opening 10.2 of the second support plate 6, and the second support plate 6 has multiple fluid-exchange apertures 11.2. Each of the first tube groups 7 is routed through a respective one of the apertures 11.2 in the second support plate 6.

The first support plate 5 supports the tubes 4 of the first tube groups 7 in the tube openings 10.1 of the first support plate 5 transversely to the longitudinal direction 9 of the tubes 4 and the second support plate 6 supports the tubes 4 of the second tube groups 8 in the tube openings 10.2 of the second support plate 8 transversely to the longitudinal direction 9 of the tubes 4.

The tubes 4 are connected at a first end 18 and at a second end 19 to a respective tube end plate. The reactor vessel 2 and the tubes 4 of the tube bundle 3 are upright. The first end 18 is open and the second end 19 is at the bottom of the reactor vessel 2.

The synthesis gas is distributed in a distributor 20 among the tubes 4 of the tube bundle 3.

The tubes 4 contain a catalyst 30. The synthesis gas contains reaction reactants. The reaction reactants for the methanol synthesis are conducted through the tubes 4 of the tube bundle 3. The synthesis gas in the tubes 4 is partially converted to methanol. A cooling medium is conducted through the reactor vessel 2 outside the tubes 4 of the tube bundle 3. The heat released from the exothermic reaction in the tube 4 is transferred via the tube wall to the cooling medium located in a shell space 24. While the catalyst 30 in the tube 4 is cooled by this process, energy is fed to the cooling medium. The cooling medium used is water, which is fed to the shell space 24 at the second end 19. Resulting bubbles of steam and the boiling two-phase water mixture flow vertically upwards owing to differences in density. The steam flows through the apertures 11.1 of the first support plate 5 and through the apertures 11.2 of the second support plate 6 with small pressure drops. The steam is collected at the top and fed to a water condenser 31. The reservoir of the condenser 31 contains saturated vapour 22 and water 23 at or slightly below boiling point. The water 23 is fed to the first device 1.1 at the second end 19 and distributed throughout the shell space 24 of the reaction vessel 2. The cooling thus works on the thermo-siphon effect.

Product gas and the partially unreacted synthesis gas are collected from the tube bundle 3 in a collector 21. The gas mixture then flows to the second device 1.2.

In the second device 1.2, which is downstream of the first device 1.1, synthesis gas from a synthesis gas inlet 25 is pre-heated.

The second device 1.2 has a partially similar structure to the first device 1.1.

By contrast to the first device 1.1, the second device 1.2 contains, between the tubes 4 of the second device 1.2 in the shell space 24, a catalyst 30 in the form of a catalyst bed.

The synthesis gas, which comprises the reaction reactants, is conducted for the methanol synthesis through the shell space 24 and through the reactor vessel 2 outside the tubes 4 of the tube bundle 3 and a cooling medium is conducted through the tubes 4 of the tube bundle 3. The synthesis gas in the shell space 24 flows downwards to a product gas outlet 28 through the apertures 11.1 of the first support plate 5 and through the apertures 11.2 of the second support plate 6 with small pressure drops.

The heat released from the exothermic reaction in the shell space 24 is transferred via the tube wall to the cooling medium located in the tube 4. While the catalyst 30 in the shell space 24 is cooled by this process, energy is fed to the cooling medium. The cooling medium used is synthesis gas, which is fed to the tubes 4 via the synthesis gas inlet 25. The energy fed in causes the synthesis gas from the synthesis gas inlet 25 to be heated and passed, in the form of pre-heated synthesis gas 26, to the gas inlet 27 of the first device 1.1.

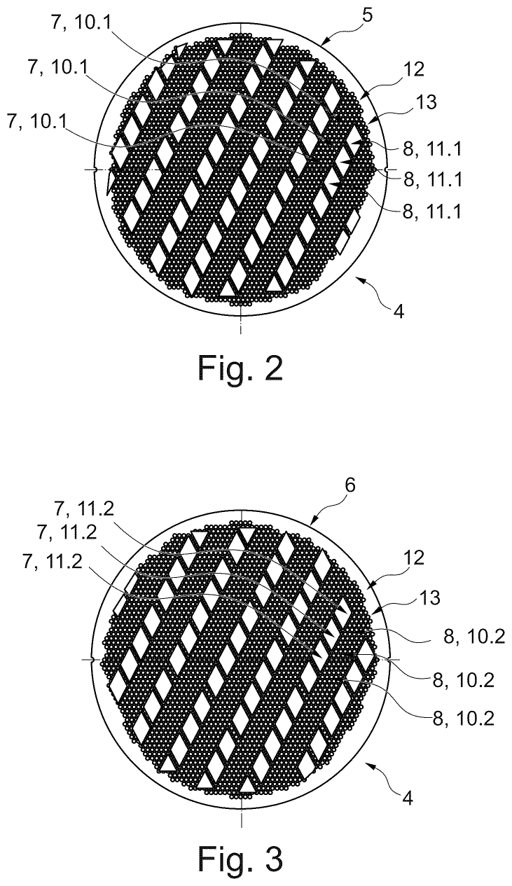

FIG. 2 shows a schematic plan view of a first configuration of a first support plate 5, as can be used in the assembly 29 from FIG. 1. The apertures 11.1 in the first support plate 5 are diamond-shaped. A respective plurality of first tube groups 7 are arranged next to one another in a first row 12. A respective plurality of second tube groups 8 are arranged next to one another in an adjacent second row 13. First rows 12 and second rows 13 alternate. The tubes 4 of the second tube groups 8 are routed through the apertures 11.1 of the first support plate 5. The tubes 4 of the first tube groups 7 are routed through the respective tube openings 10.1 of the first support plate 5. A fluid exchange is enabled through the apertures 11.1 in the remaining free flow cross section between the tubes 4 of the second tube groups 8.

FIG. 3 shows a schematic plan view of a first configuration of a second support plate 6, as can be used in the assembly 29 from FIG. 1. The apertures 11.2 in the second support plate 6 are diamond-shaped. A respective plurality of second tube groups 8 are next to one another in a second row 13. A respective plurality of first tube groups 7 are next to one another in an adjacent first row 12. First rows 12 and second rows 13 alternate. The tubes 4 of the first tube groups 7 are routed through the apertures 11.2 of the second support plate 6. The tubes 4 of the second tube groups 8 are routed through the respective tube openings 10.2 of the second support plate. A fluid exchange is enabled through the apertures 11.2 in the remaining free flow cross section between the tubes 4 of the first tube groups 7.

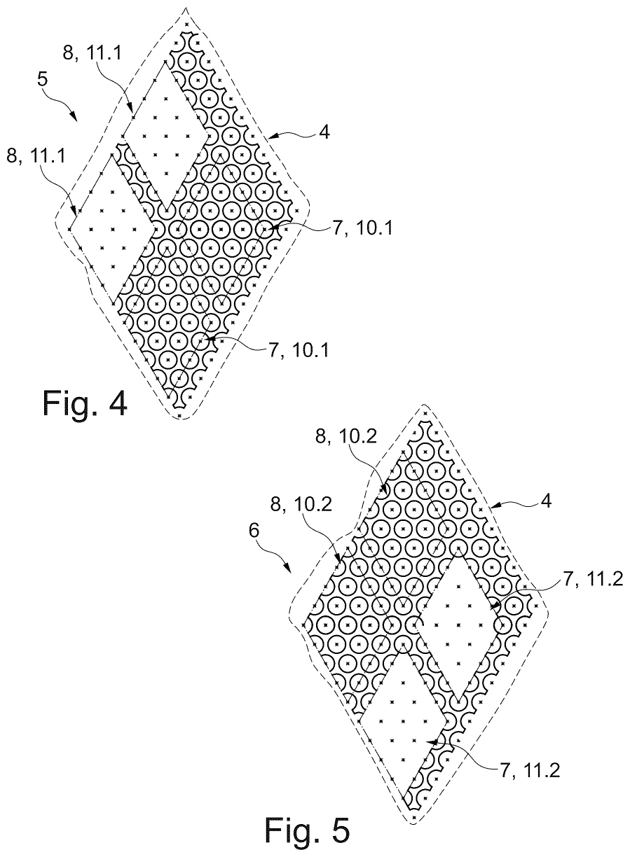

FIG. 4 shows a view of a detail of a first support plate 5 and FIG. 5 shows a view of a detail of a second support plate 6 in a first configuration from FIG. 2 and

FIG. 3. The apertures 11.1 in the first support plate 5 and the apertures 11.2 in the second support plate 6 are diamond-shaped. A respective plurality of first tube groups 7 are next to one another. A respective plurality of second tube groups 8 are next to one another. The tubes 4 of the second tube groups 8 are routed through the apertures 11.1 of the first support plate 5. The tubes 4 of the first tube groups 7 are routed through the respective tube openings 10.1 of the first support plate 5.

The tubes 4 of the first tube groups 7 are routed through the apertures 11.2 of the second support plate 6. The tubes 4 of the second tube groups 8 are routed through the respective tube openings 10.2 of the second support plate 6.

In FIG. 4, a fluid exchange is enabled through the apertures 11.1 in the remaining free flow cross section between the tubes 4 of the second tube groups 8. In FIG. 5, a fluid exchange is enabled through the apertures 11.2 in the remaining free flow cross section between the tubes 4 of the first tube groups 7. In this configuration, the diamond-shaped apertures 11.1 each comprise 5 by 5 tubes of the second tube groups 8 and the diamond-shaped apertures 11.2 each comprise 5 by 5 tubes of the first tube groups 7.

FIG. 6 shows a schematic plan view of a first support plate 5 with apertures 11.1 and of a second support plate 6 with apertures 11.2 from a superimposed perspective, in a first configuration from FIG. 2 to FIG. 5, as can be used in the assembly 29 from FIG. 1.

From a superimposed perspective, only the apertures 11.1, 11.2 of the first and of the second support plate 5, 6 are considered. The first support plate 5 and the second support plate 6 are represented in superimposed fashion in a plane for illustrative purposes. From a superimposed perspective, accordingly, a respective plurality of apertures 11.2 of the second support plate 6 are next to one another in a first row 12, wherein a respective plurality of apertures 11.1 of the first support plate 5 are next to one another in an adjacent second row 13, wherein first and second rows 12, 13 alternate. This may also be referred to as a pattern.

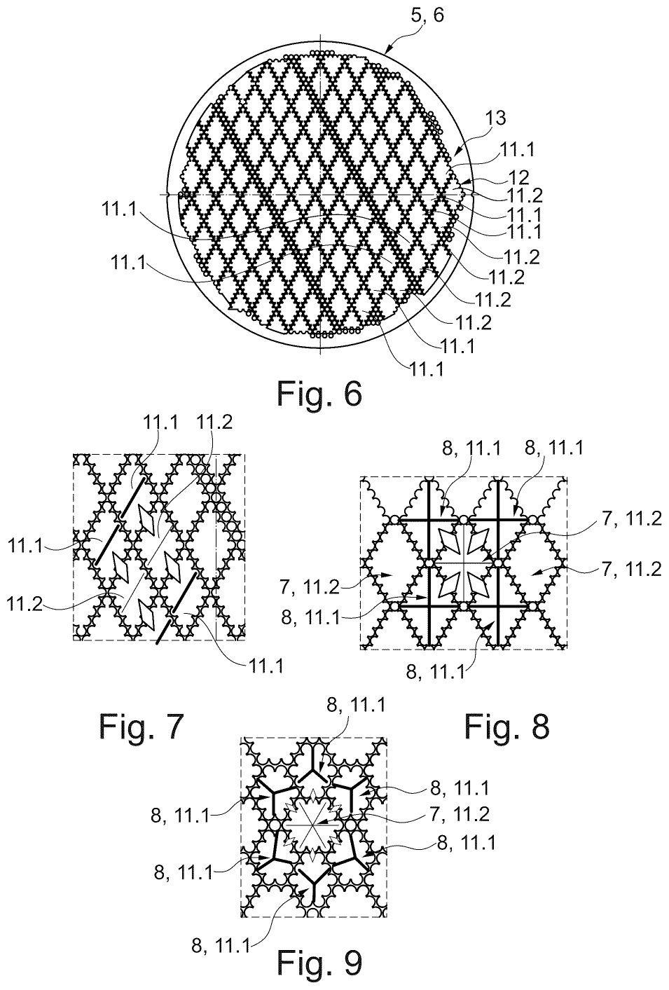

FIG. 7 shows a view of a detail of the first configuration of the apertures 11.1 of the first support plate 5 and the apertures 11.2 of the second support plate 6 from a superimposed perspective from FIG. 6. For the fluid exchange, a fluid flows through the apertures 11.1 of the first support plate 5 to the closest apertures 11.2 of the second support plate 6. As an alternative, a fluid for fluid exchange flows through the apertures 11.2 of the second support plate 6 to the closest apertures 11.1 of the first support plate 5.

FIG. 8 shows a view of a detail of a second configuration of the apertures 11.1 of the first support plate 5 and the apertures 11.2 of the second support plate 6 from a superimposed perspective, as can be used in the assembly 29 from FIG. 1. In a second configuration of the apertures 11.1, 11.2, the apertures 11.1 in the first support plate 5 each have a hexagonal shape and/or the apertures 11.2 in the second support plate 6 each have a hexagonal shape.

In this second configuration, the hexagonal apertures 11.1 each comprise 34 tubes of the second tube groups 8 and the hexagonal apertures 11.2 each comprise 34 tubes of the first tube groups 7. The respective hexagonal shape is elongate, with 2 short, opposite sides and 4 long sides.

The first tube groups 7 and second tube groups 8 alternate in a chequerboard pattern. In this pattern, four second tube groups 8 surround a first tube group 7 on the long sides of the hexagon. Accordingly, from a superimposed perspective, a respective plurality of apertures 11.2 of the second support plate 6 together with a plurality of apertures 11.1 of the first support plate 5 may alternate in a chequerboard pattern.

For the fluid exchange, a fluid flows through the apertures 11.1 of the first support plate 5 to the closest apertures 11.2 of the second support plate 6. As an alternative, a fluid for fluid exchange flows through the apertures 11.2 of the second support plate 6 to the closest apertures 11.1 of the first support plate 5.

FIG. 9 shows a view of a detail of a third configuration of the apertures 11.1 of the first support plate 5 and the apertures 11.2 of the second support plate 6 from a superimposed perspective, as can be used in the assembly 29 from FIG. 1. A respective tube group 7 is surrounded by six second tube groups 8. Accordingly, from a superimposed perspective, six apertures 11.1 of the first support plate 5 for passage of the six second tube groups 8 are disposed around the one aperture 11.2 of the second support plate 6 for passage of the one first tube group 7.

For the fluid exchange, a fluid flows through the apertures 11.1 of the first support plate 5 to the closest apertures 11.2 of the second support plate 6. As an alternative, a fluid for fluid exchange flows through the apertures 11.2 of the second support plate 6 to the closest apertures 11.1 of the first support plate 5.

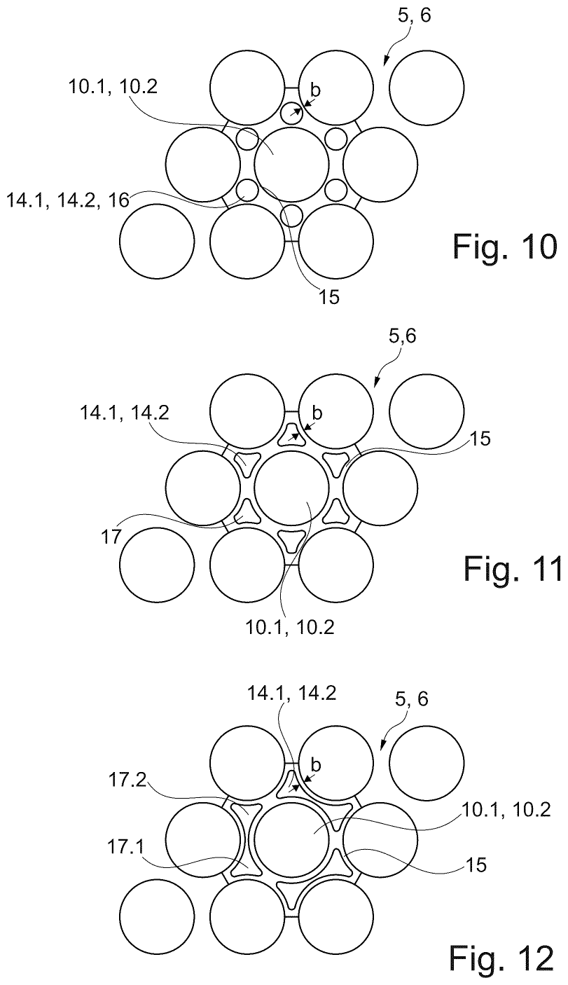

FIG. 10 shows a view of a detail of a fourth configuration of the first support plate 5 and a second support plate 6, as can be used in the devices 1.1; 1.2 from FIG. 1. The support plates 5, 6 form, for at least some cutouts 14.1, 14.2, a respective divider 15 between the cutout 14.1, 14.2 and the closest of the tube openings 10.1, 10.2 to the cutout. The apertures 14.1 are assigned to the first support plate 5. The apertures 14.2 are assigned to the second support plate 6. The dividers 15 have a respective minimum width b, which is the same for at least some of the dividers 15. The cutouts 14.1, 14.2 each lie centrally between three tube openings 10.1, 10.2 that are adjacent to one another in pairs. The cutouts 14.1, 14.2 are produced as passage bores 16.

FIG. 11 shows a view of a detail of a third configuration of a first support plate 5 and a second support plate 6, as can be used in the assembly 29 from FIG. 1. The first support plate 5 and the second support plate 6 form, for at least some cutouts 14.1, 14.2, a respective divider 15 between the cutout 14.1, 14.2 and the closest of the tube openings 10.1, 10.2 to the cutout. The apertures 14.1 are assigned to the first support plate 5. The apertures 14.2 are assigned to the second support plate 6. The dividers 15 have a respective minimum width b, which is the same for all the dividers 15. The cutouts 14.1, 14.2 each lie centrally between three tube openings 10.1, 10.2 that are adjacent to one another in pairs. The first support plate 5 and the second support plate 6 have intermediate regions 17, which are each formed between three tube openings 10.1, 10.2 that are adjacent to one another in pairs. The cutouts 14.1, 14.2 of the first support plate 5 and of the second support plate 6 are restricted to a respective one of the intermediate regions 17. The intermediate regions 17 are star-shaped.

FIG. 12 shows a view of a detail of a fourth configuration of a first support plate 5 and a second support plate 6, as can be used in the assembly 29 from FIG. 1. The first support plate 5 and the second support plate 6 form, for at least some cutouts 14.1, 14.2, a respective divider 15 between the cutout 14.1, 14.2 and the closest of the tube openings 10.1, 10.2 to the cutout. The apertures 14.1 are assigned to the first support plate 5. The apertures 14.2 are assigned to the second support plate 6. The dividers 15 have a respective minimum width b, which is the same for all the dividers 15.

The first support plate 5 and the second support plate 6 have intermediate regions 17.1; 17.2, which are each formed between three tube openings 10.1, 10.2 that are adjacent to one another in pairs, and wherein at least some of the cutouts 14.1, 14.2 of the first support plate 5 and of the second support plate 6 each extend across at least two adjacent intermediate regions 17.1; 17.2. The intermediate regions 17.1, 17.2 are star-shaped.

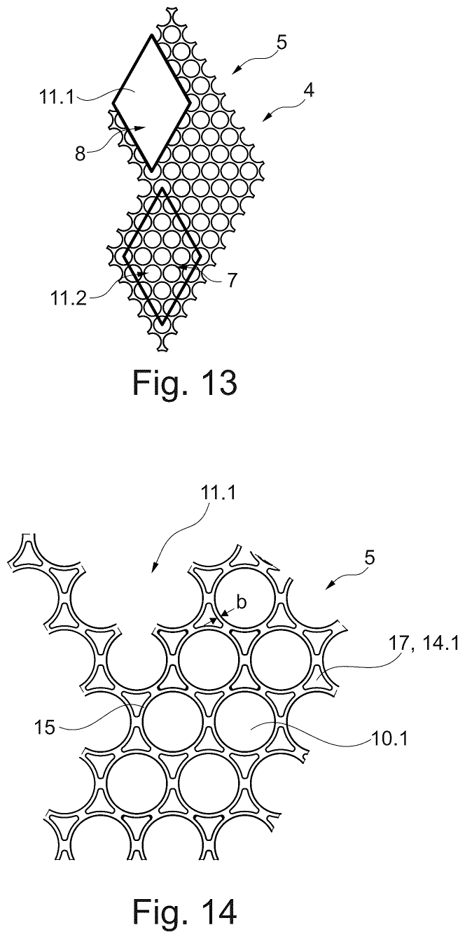

FIG. 13 and FIG. 14 show a view of a detail of a seventh configuration of the apertures and the cutouts of the first support plate 5, as can be used in the assembly 29 from FIG. 1.

The first support plate 5 forms, for at least some cutouts 14.1, a respective divider 15 between the cutout 14.1 and the closest of the tube openings 10.1 to the cutout. The dividers 15 have a respective minimum width b, which is the same for all the dividers 15. The cutouts 14.1 each lie centrally between three tube openings 10.1 that are adjacent to one another in pairs. The first support plate 5 has intermediate regions 17, which are each formed between three tube openings 10.1 that are adjacent to one another in pairs. The cutouts 14.1 of the first support plate 5 are restricted to a respective one of the intermediate regions 17. The intermediate regions 17 are star-shaped.

The apertures 11.1 in the first support plate 5 are diamond-shaped. The diamond-shaped apertures 11.2 of the second support plate 6 are only indicated. A respective plurality of first tube groups 7 are next to one another. A respective plurality of second tube groups 8 are next to one another. The tubes 4 of the second tube groups 8 are routed through the apertures 11.1 of the first support plate 5. The tubes 4 of the first tube groups 7 are routed through the respective tube openings 10.1 of the first support plate 5.

A fluid exchange is enabled through the apertures 11.1 in the remaining free flow cross section between the tubes 4 of the second tube groups 8. In this configuration, the diamond-shaped apertures 11.1 each comprise 5 by 5 tubes of the second tube groups 8.

### LIST OF REFERENCE NUMERALS

1.1; 1.2 Device
2 Reactor vessel
3 Tube bundle
4 Tube
5 First support plate
6 Second support plate
7 First tube groups
8 Second tube groups
9 Longitudinal axis
10.1; 10.2 Tube openings
11.1; 11.2 Apertures
12 First row
13 Second row
14.1; 14.2 Cutout
15 Divider
16 Passage bore
17; 17.1; 17.2 Intermediate region
18 First end
19 Second end
20 Distributor
21 Collector
22 Saturated steam
23 Water slightly below or at boiling point
24 Shell space
25 Synthesis gas inlet
26 Pre-heated synthesis gas
27 Gas inlet
28 Product gas outlet
29 Assembly
30 Catalyst
31 Condenser
b Minimum width

1. A device (1.1; 1.2) comprising a reactor vessel (2), a tube bundle (3) of multiple tubes (4), a first support plate (5) and a second support plate (6),
wherein the tube bundle (3) is located in the reactor vessel (2),
wherein the tube bundle (3) comprises multiple first tube groups (7) and multiple second tube groups (8),
wherein the first support plate (5) and the second support plate (6) are disposed in the reactor vessel (2) transversely to a longitudinal axis (9) of the reactor vessel (2),
wherein the first support plate (5) is offset from the second support plate (6) along the longitudinal axis (9) of the reactor vessel (2),
wherein each of the tubes (4) of the first tube groups (7) is routed through a respective tube opening (10.1) of the first support plate (5), and wherein the first support plate (5) has multiple fluid-exchange apertures (11.1), wherein each of the second tube groups (8) is routed through a respective one of the apertures (11.1) in the first support plate (5),
wherein each of the tubes (4) of the second tube groups (8) is routed through a respective tube opening (10.2) of the second support plate (6), and wherein the second support plate (6) has multiple fluid-exchange apertures (11.2), wherein each of the first tube groups (7) is routed through a respective one of the apertures (11.2) in the second support plate (6),
wherein the first support plate (5) supports the tubes (4) of the first tube groups (7) in the tube openings (10.1) of the first support plate (5) transversely to the longitudinal direction of the tubes (4) and the second support plate (6) supports the tubes (4) of the second tube groups (8) in the tube openings (10.2) of the second support plate (6) transversely to the longitudinal direction of the tubes (4).

2. The device (1.1; 1.2) according to claim 1, wherein the apertures (11.1) in the first support plate (5) are diamond-shaped and/or the apertures (11.2) in the second support plate (6) are diamond-shaped.

3. The device (1.1; 1.2) according to claim 1,
wherein a respective plurality of first tube groups (7) are next to one another in a first row (12), wherein a respective plurality of second tube groups (8) are next to one another in an adjacent second row (13), wherein first and second rows (12; 13) alternate.

4. The device (1.1; 1.2) according to claim 1, wherein the first support plate (5) has, in addition to the apertures (11.1), fluid-exchange cut-outs (14.1) and/or the second support plate (6) has, in addition to the apertures (11.2), fluid-exchange cut-outs (14.2).

5. The device (1.1; 1.2) according to claim 4, wherein the first support plate (5), for at least some of the cut-outs (14.1) of the first support plate (5), forms a respective divider (15) in each case between the cutout (14.1) and the closest tube opening (10.1) of the first support plate (5) to the cut-out, and wherein the dividers (15) have a respective minimum width (b) which is the same for at least some of the dividers (15),
and/or
the second support plate (6), for at least some of the cut-outs (14.2) of the second support plate (6), forms a respective divider (15) in each case between the cut-out (14.2) and the closest tube opening (10.2) of the second support plate (6) to the cut-out, and wherein the dividers (15) have a respective minimum width (b) which is the same for at least some of the dividers (15).

6. The device (1.1; 1.2) according to claim 4, wherein at least some of the cut-outs (14.1) of the first support plate (5) each lie centrally between three tube openings (10.1) of the first support plate (5) that are adjacent to one another in pairs,
and/or
at least some of the cut-outs (14.2) of the second support plate (6) each lie centrally between three tube openings (10.2) of the second support plate (6) that are adjacent to one another in pairs.

7. The device (1.1; 1.2) according to claim 4, wherein the first support plate (5) has intermediate regions (17.1; 17.2), which are each formed between three tube openings (10.1) that are adjacent to one another in pairs, and wherein at least some of the cut-outs (14.1) of the first support plate (5) each extend across two of the intermediate regions (17.1; 17.2),
and/or
wherein the second support plate (6) has intermediate regions (17.1; 17.2), which are each formed between three tube openings (10.2) that are adjacent to one another in pairs, and wherein at least some of the cut-outs (14.2) of the second support plate (6) each extend across two of the intermediate regions (17.1; 17.2).

8. A process for methanol synthesis using a device (1.1; 1.2) according to claim 1, wherein reaction reactants for the methanol synthesis are conducted through the tubes (4) of the tube bundle (3) and a cooling medium is conducted through the reactor vessel (2) outside the tubes (4) of the tube bundle (3),
or
wherein reaction reactants for the methanol synthesis are conducted through the reactor vessel (2) outside the tubes (4) of the tube bundle (3) and a cooling medium is conducted through the tubes (4) of the tube bundle (3).
